(19) United States
(12) Patent Application Publication
(10) Pub. No.: US 2025/0017466 A1
(43) Pub. Date: Jan. 16, 2025

(54) DEVICE COMMUNICATION MANAGEMENT IN USER ACTIVITY MONITORING SYSTEMS

(71) Applicant: T.J.Smith and Nephew,Limited, Hull (GB)

(72) Inventors: Allan Kenneth Frazer Grugeon Hunt, Beverley (GB); Johannes Dagevos van Rij, Skidby (GB)

(21) Appl. No.: 18/710,384

(22) PCT Filed: Nov. 22, 2022
(86) PCT No.: PCT/EP2022/082855

§ 371 Date: May 15, 2024

(30) Foreign Application Priority Data
Nov. 24, 2021 (GB) 2116900.8

(51) Int. Cl. A61B 5/00; A61B 5/11; H04W 12/50; H04W 84/18

(52) U.S. Cl. CPC A61B 5/0004; A61B 5/1118; A61B 5/447; A61B 5/6812; H04W 12/50; H04W 84/18

(57) ABSTRACT

In some aspects, a method is disclosed for managing communication between a first electronic device and a second electronic device. The method includes generating, by a sensor of the first electronic device, a first signal indicative of an ambient environment around the first electronic device; receiving, by the first electronic device, a second signal from a second electronic device or a third electronic device; comparing, by the first electronic device, the first signal to the second signal; determining, by the first electronic device, that the first signal and the second signal match; and enabling, by the first electronic device, communication between the first electronic device and the second electronic device responsive to the first electronic device determining that the first signal and the second signal match.

### CROSS-REFERENCE TO RELATED APPLICATIONS

This application claims priority to U.K. Provisional Application No. 2116900.8 filed on Nov. 24, 2021; the disclosure of which is hereby incorporated by reference in its entirety.

### BACKGROUND

### Field

Embodiments of the present disclosure relate to apparatuses, systems, and methods for managing communication between user activity monitoring devices and data processing devices.

### Description of Related Art

Pressure ulcers, which may also be known as pressure sores, bedsores, or decubitus ulcers, are injuries to skin and underlying tissue resulting from prolonged pressure on the skin, soft tissue, muscle, or bone above capillary filling pressure (approximately 32 mmHg).

One type of pressure ulcer that develops on a foot is known as a diabetic foot ulcer (DFU), which tends to occur with a higher frequency and intensity in the diabetic population. Management and treatment of diabetic foot ulcers requires offloading the diabetic foot ulcers by using cushioned footwear, such as a support boot, cast, shoe, or the like. While offloading can be effective, it has been found that non-compliance with or non-use of the offloading devices can play a large role in the delayed healing of the diabetic foot ulcers.

Prior art approaches and systems provide little or no information regarding an individual's lifestyle and compliance with the offloading devices. Gaining insight into the individual's lifestyle can be important for the prevention and healing of pressure ulcers. However, because of these limitations, the prevention and healing of pressure ulcers using prior art approaches and systems may be delayed or, worse yet, worsened leading to prolonged discomfort, hospitalization, or even surgery.

### BRIEF DESCRIPTION OF THE DRAWINGS

Embodiments of the present disclosure will now be described hereinafter, by way of example only, with reference to the accompanying drawings in which:

FIG. 1A illustrates a user activity monitoring system including an example activity monitoring device;

FIG. 1B illustrates the activity monitoring device of FIG. 1A in communication with an example data processing device;

FIG. 1C illustrates the activity monitoring device and the data processing device of FIG. 1B in communication with another example data processing device;

FIG. 2 illustrates example components of the activity monitoring device of FIGS. 1A through 1C and the data processing device(s) of FIGS. 1B and 1C;

FIG. 3A illustrates an example device communication initiation process;

FIG. 3B illustrates another example device communication initiation process;

FIG. 4 illustrates yet another example device communication initiation process;

FIG. 5A illustrates an example of a match between signals; and

FIG. 5B illustrates an example of a non-match between signals.

### DETAILED DESCRIPTION

### Introduction

Securely pairing devices can be challenging when one or more of the devices to be paired lack a user input method (for example, keyboard/buttons) or a visual output (for example, screen or LEDs). A secure pairing may involve devices confirming the identity of the other devices. It can be desirable in certain implementations that a user confirmation is made on a single device with a user input to ensure that the device initiates a pairing with an intended device. This may be achieved by pushing a button on the intended device.

In one example, two devices, such as an activity monitoring device and a data processing device, in the neighboring vicinity of one another may be paired. Both devices may be configured to monitor an electromagnetic frequency band (for example, RF (radio frequency) frequency band) for activity. This frequency band may be agreed upon between the two devices. The timers of each device may be synchronized. The devices may then each take a sample of the background electromagnetic activity (for example, RF activity) present in the agreed upon electromagnetic frequency band. A comparison of the samples can be used to determine that the devices were in the same location.

Two devices may agree on a modification to a background (for example, an electromagnetic background, a sound background, a pressure background, or a temperature background) near the two devices. One device, for instance, can generate a modification in an electromagnetic background that may serve to encrypt a signal communicated via the electromagnetic background. The other device may monitor the electromagnetic background to generate a signal and then account for an impact of the modification on the signal. The other device may thus effectively decrypt the signal communicated via the electromagnetic background by accounting for the impact of the modification.

In some implementations, one device can generate an agreed modification of the background RF environment (which may effectively encrypt a communication using the background RF environment as a shared key), and another device may decrypt the communication if the another device possesses the same background key. In some implementations, one or more devices can transmit a signal to a background to artificially generate a temporary environment to facilitate communication configuration or transmission of data.

A third device can be used in some instances to dictate the electromagnetic frequency band that is to be used. The third device can generate and transmit the electromagnetic signature (for example, RF signature) in that band. Both pairing devices, which may be positioned in a vicinity of the third device, then may detect and compare the received samples against a signature template provided by the third device.

A third device can, in some instances, can be dictated a frequency band that has been agreed by the two pairing devices. The third device may then generate the electromagnetic signature in that band. Both pairing devices can then compare their received samples within the frequency band against a signature template provided by the third device.

A third device can, in some instances, perform a comparison of samples from the pairing devices against the signature template.

### Overview

Some aspects disclosed herein relate to apparatuses, systems, and methods for using an ambient environment around devices (for example, electromagnetic radiation, sound, or another detectable signal in the ambient environment) to securely arrange in-band communication (for example, via radio waves) between an activity monitoring device and a data processing device. Once in-band communication between the activity monitoring device and the data processing device has been setup by comparing signals indicative of ambient environments around the activity monitoring device and the data processing device, the activity monitoring device can, for example, transmit data gathered by the activity monitoring device to the data processing device via in-band communication, and the data processing device can transmit via in-band communication commands to the activity monitoring device, such as for adjusting operations of the activity monitoring device. The data gathered by the activity monitoring device can advantageously, in certain aspects, be used to prevent, monitor, or otherwise manage pressure ulcers for a user of the activity monitoring device.

In one example, the activity monitoring device and the data processing device can each generate a signal indicative of their ambient environment. The activity monitoring device can transmit its signal to the data processing device, and the data processing device can receive this signal and compare the signal to its own signal. The activity monitoring device can determine that its own signal matches the signal from the data processing device to enable secure communication between the devices, such as via radio waves using a protocol like Bluetooth™ Low Energy. Similarly, the data processing device can determine that its own signal matches the signal from the activity monitoring device to enable secure communication between the devices.

Although an activity monitoring device and a data processing device are used as examples to describe certain features described herein, at least some of the features can apply more broadly to communication between two or more electronic devices that may perform one or more additional or alternative functions than those described for an activity monitoring device and a data processing device.

One or more features disclosed herein can, in certain aspects, desirably help prevent a man-in-the-middle attack where an attacker may secretly relay and possibly alter communication between two electronic devices that believe they are directly communicating with each other. The one or more features can at least help prevent the man-in-the-middle attack by assisting with establishing a secure communication between the two electronic devices where it may be very difficult for the attacker to circumvent mutual authentication and for the attacker to impersonate each of the two electronic devices.

### User Activity Monitoring

Activities of a user may be desirably monitored by an activity monitoring device for a variety of reasons, including wound prevention and monitoring. In one example, the activities of a user can be monitored when the user may be prone to or already have a wound, such as a pressure ulcer. Information gathered by the activity monitoring device about the activities of the user can be helpful for assisting with prevention or treatment of the pressure ulcer. In addition, information gathered by the activity monitoring device about the activities can be useful for checking compliance with a treatment regimen.

Some aspects disclosed herein relate to wound monitoring or therapy for a human or animal body. Therefore, any reference to a wound herein can refer to a wound on a human or animal body, and any reference to a body herein can refer to a human or animal body. The disclosed technology may relate to preventing or minimizing damage to physiological tissue or living tissue, or to the treatment of damaged tissue (for example, a wound as described herein).

As used herein the expression “wound” may include an injury to living tissue may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound can become a chronic wound when it does not follow the healing stages resulting in a lengthened recovery. It is believed that the transition from acute to chronic wound can be due to a patient being immuno-compromised.

Chronic wounds may include for example: venous ulcers (such as those that occur in the legs), which account for the majority of chronic wounds and mostly affect the elderly, diabetic ulcers (for example, foot or ankle ulcers), peripheral arterial disease, pressure ulcers, or epidermolysis bullosa (EB).

Examples of other wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Wounds may include a deep tissue injury. Deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

Wound may include tissue at risk of becoming a wound as discussed herein. For example, tissue at risk may include tissue over a bony protuberance (at risk of deep tissue injury/insult) or pre-surgical tissue (for example, knee tissue) that may have the potential to be cut (for example, for joint replacement/surgical alteration/reconstruction).

Some aspects relate to methods of monitoring or treating a wound with the technology disclosed herein in conjunction with one or more of the following: advanced footwear, turning a patient, offloading (such as, offloading diabetic foot ulcers), treatment of infection, systemics, antimicrobial, antibiotics, surgery, removal of tissue, affecting blood flow, physiotherapy, exercise, bathing, nutrition, hydration, nerve stimulation, ultrasound, electrostimulation, oxygen therapy, microwave therapy, active agents ozone, antibiotics, antimicrobials, or the like.

Alternatively or additionally, a wound may be treated using topical negative pressure or traditional advanced wound care, which is not aided by the using of applied negative pressure (may also be referred to as non-negative pressure therapy).

Although the present disclosure may refer to pressure ulcers, foot ulcers, or the like, the systems and methods disclosed herein can be used for preventing, monitoring, or treating any type of skin injury or wound, such as a venous leg ulcer.

### User Activity Monitoring System

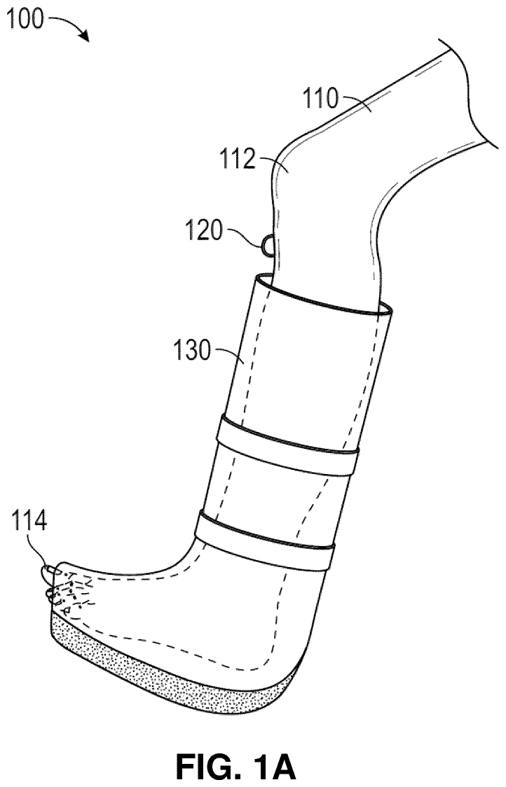

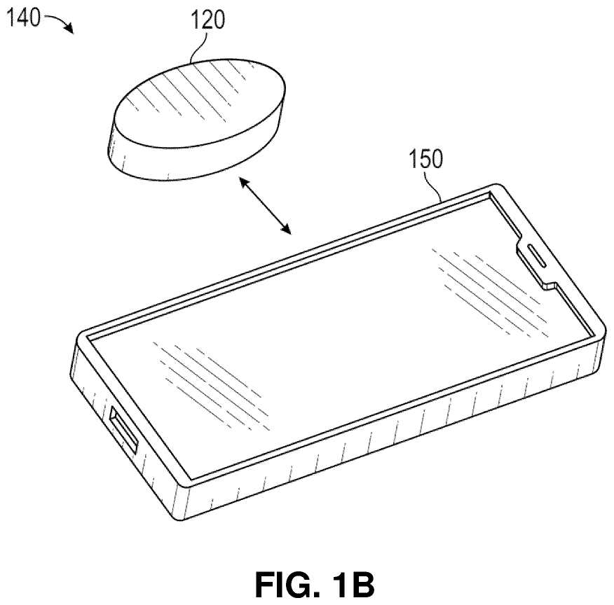

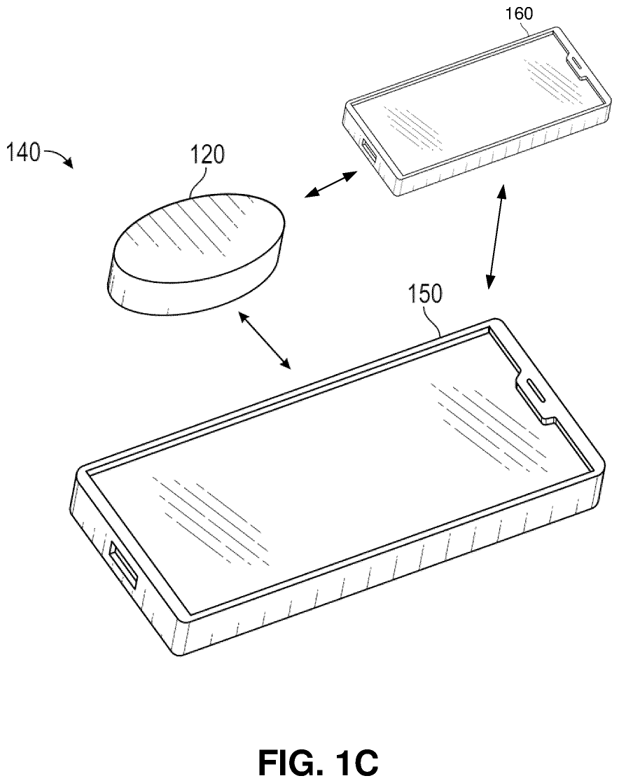

FIG. 1A illustrates a user activity monitoring system 100 including an activity monitoring device 120 attached to a body part 110. The activity monitoring device 120 can be attached to the body part 110 using a strap, adhesive, or other coupling mechanism and may be worn on or supported by the body.

The body part 110 can be a leg of a user that includes a knee 112 and a foot 114. As illustrated the activity monitoring device 120 can be supported by the body part 110 at a position between the knee 112 and the foot 114, such as proximate to the knee 112. In other aspects, the activity monitoring device 120 can be supported by another part of the body part 110, such as above the knee 112 or elsewhere. The activity monitoring device 120 can be supported using a strap, adhesive, or other coupling mechanism. The activity monitoring device 120 can monitor and record activities (for instance, walking, jumping, sitting, laying down, running, squatting, or standing) of the body part 110, such as from a position, movement, or orientation of the activity monitoring device 120 or one or more other sensors of the activity monitoring device 120. The activity monitoring device 120 can, for example, be used for loading monitoring of loading of the foot 114. In certain aspects, multiple body parts can be monitored by the activity monitoring device 120, and different sensors can be used for monitoring different body parts. The activity monitoring device 120 can include a sensor configured to generate a signal indicative of an ambient environment around the activity monitoring device 120, such as electromagnetic radiation, sound, pressure, or temperature in the ambient environment.

The body part 110 is shown wearing and being partly covered by an orthopedic device 130 (sometimes referred to as an offloading device). The orthopedic device 130 can support the body part 110 and reduce a pressure on the foot 114 when the user may be standing or engaging in other activities.

Although not illustrated in FIG. 1A, the user activity monitoring system 100 can additionally or alternatively include one or more of the activity monitoring device 120 at other positions, such as at a position supported by the orthopedic device 130, another part of the body part 110, another device not worn such as a cane or a walker, or elsewhere. These one or more additional or alternative of the activity monitoring device 120 can be the same as or similar to the activity monitoring device 120 and may monitor and record activities of the orthopedic device 130, the another part of the body part 110, or the body.

FIG. 1B illustrates a data transfer system 140 that includes the activity monitoring device 120 and a data processing device 150. The activity monitoring device 120 can be positioned proximate to the data processing device 150 and communicate, such as wirelessly, with the data processing device 150.

The activity monitoring device 120 and the data processing device 150 can together configure a communication channel with one another to permit transfer of recorded activities or other data from the activity monitoring device 120 and the data processing device 150 or transfer of one or more commands from the data processing device 150 to the activity monitoring device 120, among other possibilities. As described herein, the activity monitoring device 120 and the data processing device 150 can configure the communication channel, such as a radio wave channel, using an ambient environment around the activity monitoring device 120 and the data processing device 150, such as electromagnetic radiation, sound, or another detectable signal in the ambient environment. The data processing device 150 can, for example, be a smart phone or a tablet computer. The data processing device 150 can include a sensor configured to generate a signal indicative of an ambient environment around the device.

FIG. 1C illustrates another example data transfer system 140 that includes the activity monitoring device 120, the data processing device 150, and a data processing device 160. The activity monitoring device 120, the data processing device 150, and the data processing device 160 can be positioned proximate to each other and communicate, such as wirelessly, with each other.

The data processing device 160 can assist or manage configuring of a communication between the activity monitoring device 120 and the data processing device 150 to permit transfer of recorded activities or other data from the activity monitoring device 120 and the data processing device 150 or transfer of one or more commands from the data processing device 150 to the activity monitoring device 120, among other possibilities. As described herein, the activity monitoring device 120, the data processing device 150, or the data processing device 160 can communicate over two or more communication mediums setup the communication channel between the activity monitoring device 120 and the data processing device 150. The data processing device 160 can include an emitter or transmitter configured to provide electromagnetic radiation, sound, or another detectable signal to the ambient environment around the data processing device 160. The data processing device 160 can include a sensor configured to generate a signal indicative of an ambient environment. The data processing device 160 can, for example, be a smart phone or a tablet computer.

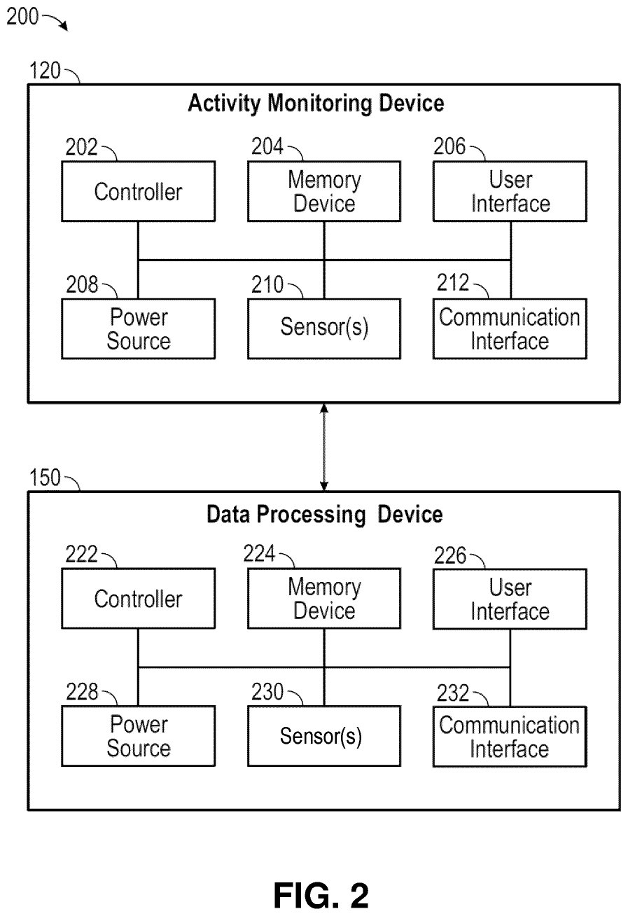

FIG. 2 illustrates components 200 of the activity monitoring device 120 and the data processing device 150. The data processing device 160 can include similar or the same components as the data processing device 150. The activity monitoring device 120 can include a controller 202, a memory device 204, a user interface 206, a power source 208, one or more sensors 210, and a communication interface 212 that are configured to communicate, such as electrically communicate, with one another. The power source 208 can provide power to one or more components of the activity monitoring device 120.

The components of the activity monitoring device 120 can be contained in or supported by a housing of the activity monitoring device 120. The housing can be composed of a top portion and a bottom portion that are sealed together, and the top portion or the bottom portion can be hard or soft. The housing can be flexible and have a mechanical structure and design features that provide for a shouldered keyway alignment of components within the housing. The housing can support a circuit board on its inside and on which one or more components of the activity monitoring device 120 may be positioned.

The data processing device 150 can include a controller 222, a memory device 224, a user interface 226, a power source 228, one or more sensors 230, and a wireless communication interface 232 that are configured to communicate, such as electrically, with one another. The power source 228 can provide power to one or more components of the data processing device 150. The components of the data processing device 150 can be contained in or supported by a housing of the data processing device 150.

In other aspects, the activity monitoring device 120 and the data processing device 150 can include additional or alternative components than those illustrated in FIG. 2.

Referring to the activity monitoring device 120, the controller 202 can control operations of one or more other components (for instance, the memory device 204, the user interface 206, the power source 208, the one or more sensors 210, or the communication interface 212) of the activity monitoring device 120 according at least to instructions stored in the memory device 204. The controller 202 can, for instance, control monitoring of loading of the body part 110 with a weight of the body or positioning of the body part 110 and record data indicative of loading of the body part 110 or positioning of the body part 110 to the memory device 204.

The user interface 206 can include one or more output elements, such as visual feedback devices (for example, light emitting diodes), haptic feedback devices, or audio devices (for example, speakers), that provide user outputs to a user. The one or more elements can convey status information to the user like whether the activity monitoring device 120 is successfully functioning or has successfully configured communication with the data processing device 150. In some aspects, the user interface 206 may not include one or more input elements, such as buttons, switches, dials, touch pads, microphones, or touch screens, for receiving user inputs for configuring the activity monitoring device 120. In such aspects, the activity monitoring device 120 can arrive to a user preconfigured so that the activity monitoring device 120 is able to function without receiving such user inputs, or the activity monitoring device 120 can be configured through communication with another device, such as the data processing device 150. In some aspects, the user interface 206 may have no more than one user input element, such as a button, for receiving user inputs to activate and deactivate the activity monitoring device 120 or performing one or more other functions.

The one or more sensors 210 can be used to monitor an ambient environment around the activity monitoring device 120 and generate a signal indicative of the ambient environment. The signal can, for example, be indicative of variations in electromagnetic radiation, sound, pressure, or temperature around the activity monitoring device 120 over a period of time. Certain signals generated by the one or more sensors 210 can be recognized by the controller 202 and trigger initiation or completion of particular functions, such as initiating or authenticating a communication pairing between the activity monitoring device 120 and the data processing device 150 to permit future communication. In addition, the one or more sensors 210 can detect and monitor activities of the user of the activity monitoring device 120 that include, for instance, a loading or positioning of the body part 110. The one or more sensors 210 can include one or more accelerometers, gyroscopes, magnetometers, impedance sensors, thermistors, pressure sensors, or optical sensors, among other types of sensors. The one or more sensors 210 can be positioned proximate to the body part 110 or may be remote from the body part 110 yet usable to monitor characteristics of the body part 110.

The communication interface 212 can be used to communicate with the data processing device 150 and the data processing device 160, such as via radio waves and according to a Bluetooth™ protocol like Bluetooth™ Low Energy or another protocol. The communication interface 212 can, for example, communicate and pair with other devices and transmit device usage or sensor data like alarms, monitored loading or positioning, or changes to a monitoring or therapy program performed by the activity monitoring device 120 to the data processing device 150. The communication interface 212 can be used to receive data, including commands, from the data processing device 150 and the data processing device 160.

The communication interface 212 can be permitted to communicate with (for example, transfer data to or process commands from) the data processing device 150 once a communication channel is configured (for example, by device pairing) between the communication interface 212 and the data processing device 150. In some implementations, the communication interface 212 may be permitted to communicate one-way with the data processing device 150 before a communication channel is configured between the communication interface 212 and the data processing device 150.

Turning to the data processing device 150, as illustrated in FIG. 2, the controller 222 of the data processing device 150 can control operations of one or more other components of the data processing device 150 according at least to instructions stored in the memory device 224. The controller 222 can, for instance, configure and control communication with the activity monitoring device 120, as well as process data received from the activity monitoring device 120 or send commands to the activity monitoring device 120. The data processing device 150 can execute one or more applications to assist with communicating with the activity monitoring device 120.

The user interface 226 of the data processing device 150 can include one or more elements that receive user inputs or provide user outputs to a user. The one or more input elements of the user interface 226 that receive user inputs can include buttons, switches, dials, touch screens, or the like, and the one or more output elements that provide user outputs can include indicators, screens, speakers, or the like. The user outputs can, for instance, indicate to a user how to enable communication between the activity monitoring device 120 and the data processing device 150 via the communication interface 212 of the activity monitoring device 120.

The one or more sensors 230 can be used to monitor an ambient environment around the data processing device 150 and generate a signal indicative of the ambient environment. The signal can, for example, be indicative of variations in electromagnetic radiation, sound, pressure, or temperature around the data processing device 150 over a period of time. Certain signals generated by the one or more sensors 230 can be recognized by the controller 222 to trigger initiation or completion of particular functions, such as initiating or authenticating a communication pairing between the activity monitoring device 120 and the data processing device 150 to permit future communication.

The wireless communication interface 232 of the data processing device 150 can be used to communicate with the activity monitoring device 120 and the data processing device 160, such as via radio waves and according to a Bluetooth™ protocol like Bluetooth™ Low Energy or another protocol. The wireless communication interface 232 can, for example, receive device usage data like alarms, monitored loading or positioning, or changes to a monitoring or therapy program performed by the activity monitoring device 120 or transmit data like commands.

### Device Communication Management

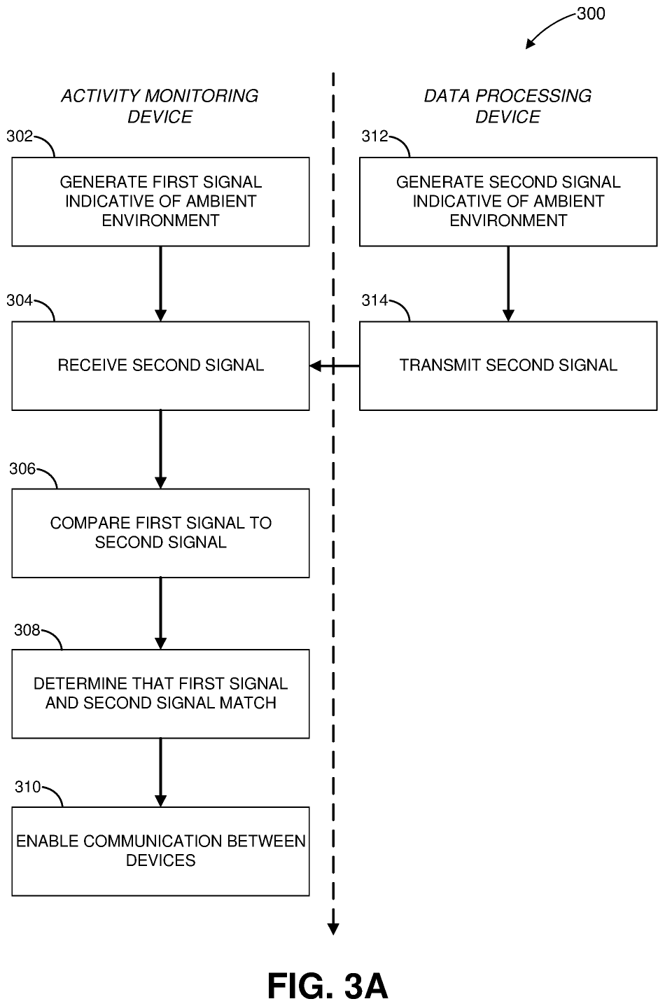

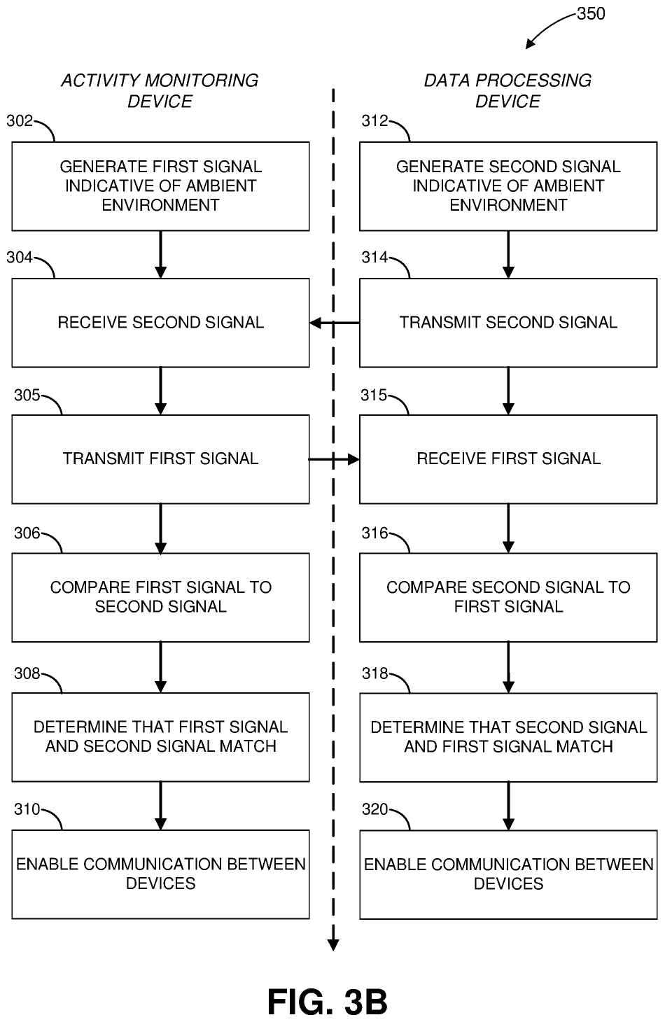

FIG. 3A illustrates a device communication initiation process 300. For convenience, the process 300 is described in the context of the user activity monitoring system 100, but may instead be implemented in other components or systems described herein, or by other computing systems not shown. The process 300 can advantageously, in certain aspects, enable the activity monitoring device 120 and the data processing device 150 to establish a secure communication channel for transmitting information, such as data collected by the activity monitoring device 120 by activities of a user of the activity monitoring device 120. Communication between the activity monitoring device 120 and the data processing device 150 can, for instance, be performed according to a Bluetooth™ Low Energy protocol once established.

At block 302, the activity monitoring device 120 can generate a first signal indicative of an ambient environment around the activity monitoring device 120. The first signal may, for example, be indicative of electromagnetic radiation, sound, pressure, or temperature in the ambient environment. At block 312, the data processing device 150 can generate a second signal indicative of an ambient environment around the data processing device 150. The second signal may, for example, be indicative of electromagnetic radiation, sound, pressure, or temperature in the ambient environment. Blocks 302 and 312 may happen simultaneously or substantially at the same time, or in any order one before or after the other. If blocks 302 and 312 happen simultaneously or substantially at the same time, the activity monitoring device 120 and the data processing device 150 may be monitoring the ambient environment simultaneously or substantially at the same time and thus detecting common variations in the ambient environment. Timers in the activity monitoring device 120 and the data processing device 150 can, for instance, be synchronized so that the activity monitoring device 120 and the data processing device 150 monitor the ambient environment simultaneously or substantially at the same time.

At block 314, the data processing device 150 can transmit the second signal to the activity monitoring device 120 via the communication interface 232. At block 304, the activity monitoring device 120 can receive the second signal via the communication interface 212.

At block 306, the activity monitoring device 120 can compare the first signal to the second signal. The activity monitoring device 120 may compare the first signal and the second signal to determine whether the first signal and the second signal have corresponding characteristics within a tolerance (for instance, a similar energy or frequency characteristic differing by less than a threshold amount). For example, the activity monitoring device 120 can compare a portion of the first signal to a portion of the second signal. In one implementation, the activity monitoring device 120 can compare the first signal within a frequency band to the second signal within the frequency band.

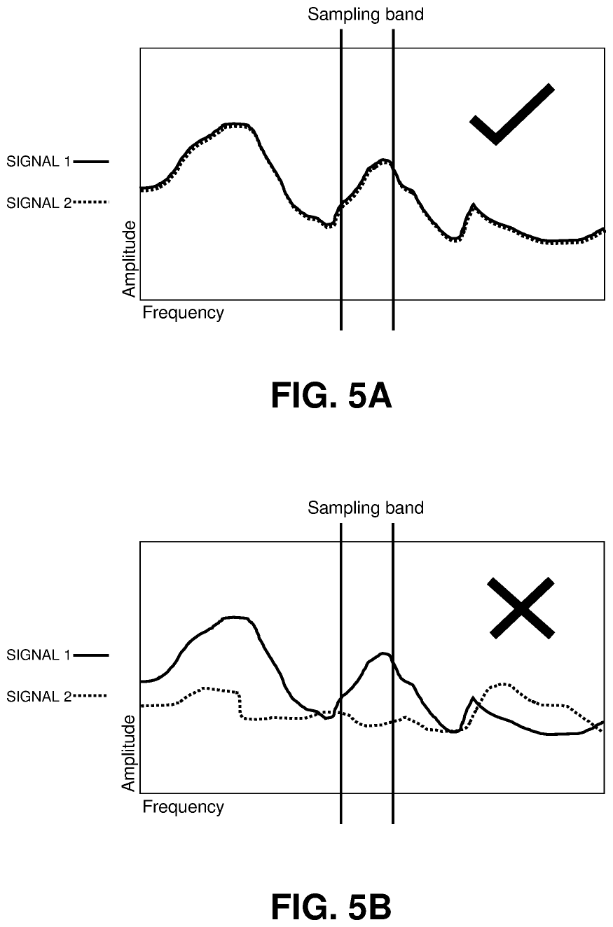

At block 308, the activity monitoring device 120 can determine that the first signal and the second signal match. For example, the activity monitoring device 120 may determine that the first signal and the second signal are the same or substantially the same (for instance, satisfy a template match criteria or have sufficiently similar waveform characteristics, such as is discussed with respect to FIGS. 5A and 5B).

At block 310, the activity monitoring device 120 can enable communication between the activity monitoring device 120 and the data processing device 150. The activity monitoring device 120 may, for instance, enable communication between the activity monitoring device 120 and the data processing device 150 responsive to the activity monitoring device 120 determining that the first signal and the second signal match. Enabling communication can include transferring authentication information, such as a key like an encryption key or a passkey, to authenticate and secure communication between the devices. Enabling communication can include pairing of the devices. As described elsewhere herein, the communication can include secure communication between devices. Further, the communication may be via radio waves using a protocol like Bluetooth™ Low Energy.

FIG. 3B illustrates a device communication initiation process 350. For convenience, the process 350 is described in the context of the user activity monitoring system 100, but may instead be implemented in other components or systems described herein, or by other computing systems not shown. Similar to the process 300, the process 350 can advantageously, in certain aspects, enable the activity monitoring device 120 and the data processing device 150 to establish a secure communication channel for transmitting information, such as data collected by the activity monitoring device 120 by activities of a user of the activity monitoring device 120. The process 350 can share many aspects of the process 300, and as such similar process blocks share common block labels. Additional blocks 305, 315, 316, 318, and 320 will be described next without repeat of blocks 302, 312, 304, 314, 306, 308, and 310 as described above relative to the process 300.

At block 305, the activity monitoring device 120 can transmit the first signal via the communication interface 212 to the data processing device 150. At block 315, the data processing device 150 can receive the first signal via the communication interface 232.

At block 316, the data processing device 150 can compare the second signal to the first signal. The data processing device 150 may compare the second signal and the first signal to determine whether the second signal and the first signal have corresponding characteristics within a tolerance (for instance, a similar energy or frequency characteristic differing by less than a threshold amount). For example, the data processing device 150 can compare a portion of the second signal to a portion of the first signal. In one implementation, the data processing device 150 can compare the first signal within a frequency band to the second signal within the frequency band. The frequency band used in block 316 may be the same frequency band used in block 306.

At block 318, the data processing device 150 can determine that the second signal and the first signal match. For example, the data processing device 150 may determine that the second signal and the first signal are the same or substantially the same (for instance, satisfy a template match criteria or have sufficiently similar waveform characteristics, such as is discussed with respect to FIGS. 5A and 5B).

At block 320, the data processing device 150 can enable communication between the data processing device 150 and the activity monitoring device 120. The data processing device 150 may, for instance, enable communication between the data processing device 150 and the activity monitoring device 120 responsive to the data processing device 150 determining that the second signal and the first signal match. Enabling communication may result in similar device setup or communication to the example setup or communications discussed with respect to block 310.

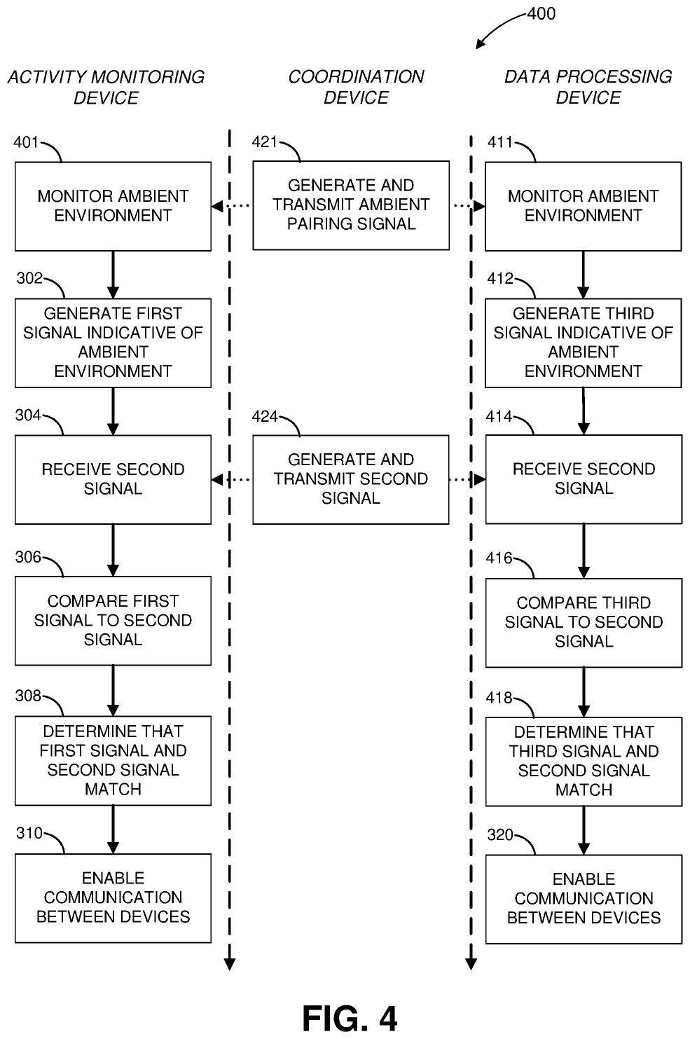

FIG. 4 illustrates a device communication initiation process 400. For convenience, the process 400 is described in the context of the user activity monitoring system 100, but may instead be implemented in other components or systems described herein, or by other computing systems not shown. The process 400 can advantageously, in certain aspects, enable the activity monitoring device 120 and the data processing device 150 to establish a secure communication channel for transmitting information, such as data collected by the activity monitoring device 120 by activities of a user of the activity monitoring device 120. The establishing of the secure communication channel can be facilitated by a coordination device, such as the data processing device 160. The process 400 shares aspects of the process 300 and the process 350, and as such similar process blocks share common block labels. As such, additional blocks 401, 411, 412, 414, 416, 418, 421, and 424, will be described next without repeat of blocks 302, 304, 306, 308, 310, and 320.

At block 421, the data processing device 160 can generate and transmit an ambient pairing signal. The ambient pairing signal may, for example, be transmitted as electromagnetic radiation, sound, pressure, or temperature in the ambient environment around the data processing device 160.

At block 401, the activity monitoring device 120 can monitor an ambient environment around the activity monitoring device 120, which can, in turn, be used at block 302 to generate the first signal indicative of the ambient environment. The first signal may vary responsive to the ambient pairing signal transmitted by the data processing device 160. Similarly, at block 411, the data processing device 150 can monitor an ambient environment around the data processing device 150, which can, in turn, be used at block 412 to generate a third signal indicative of the ambient environment. The third signal may vary responsive to the ambient pairing signal transmitted by the data processing device 160. The operation at block 412 can be similar to the operation at block 312 except that the third signal may be generated rather than the second signal.

The generating of the first signal by the activity monitoring device 120 at block 302 can occur simultaneously with the generating of the third signal by the data processing device 150 at block 412. The generating and transmitting of the ambient pairing signal by the data processing device 160 at block 421 to the activity monitoring device 120 can occur simultaneously or substantially at the same time or before or after generating and transmitting of the ambient pairing signal by the data processing device 160 at block 421 to the data processing device 150.

At block 424, the data processing device 160 can generate and transmit a second signal to the activity monitoring device 120 and the data processing device 150. The generating and transmitting of the second signal by the data processing device 160 at block 424 to the activity monitoring device 120 can occur simultaneously or substantially at the same time or before or after generating and transmitting of the second signal by the data processing device 160 at block 424 to the data processing device 150.

In turn, the activity monitoring device 120 can receive the second signal at block 304, and the data processing device 150 can receive the second signal at block 414. The operation at block 414 can be similar to the operation at block 314 except that the second signal may be received rather than the first signal. The activity monitoring device 120 and the data processing device 150 can receive the second signal simultaneously or substantially at the same time, or in any order one before or after the other. In some implementations, the second signal can include an identification of what portion (for instance, a frequency band) of the first signal or the third signal generated at blocks 302 and 412 are to be used for comparing at blocks 306 and 416. The operations at block 416 and 418 can respectively be similar to the operations at blocks 316 and 318 except that the second signal in blocks 416 and 418 may be used in place of the first signal in blocks 316 and 318 and that the third signal in blocks 416 and 418 may be used in place of the second signal in blocks 316 and 318.

FIGS. 5A and 5B illustrate examples of a match and a non-match, respectively, between signals indicative of an ambient environment around devices. FIGS. 5A and 5B each show signal frequency on the x-axis and signal amplitude on the y-axis. The signals may be data samples indicative of electromagnetic radiation, sound, pressure, or temperature (among other possibilities) in the ambient environment around devices. Also shown in both FIGS. 5A and 5B is a sampling band that may define a portion of the signals used for comparison. In some implementations, other sampling bands or entire portions of signals may be compared.

As shown in FIG. 5A, the first signal and the second signal, as assessed within the sampling band shown, can match. Also shown, the first signal and the second signal outside the sampling band also may match.

As shown in FIG. 5B, the first signal and the second signal, as assessed within the sampling band shown, may not match. Also shown, the first signal and the second signal outside the sampling band also may not match.

Determining a match or non-match between signals can include determining whether a portion of the signals, such as within a sampling band as shown in FIGS. 5A and 5B, have corresponding characteristics within a tolerance (for instance, similar amplitudes or standard deviations, among other possibilities). A tolerance may be, in some implementations, within about 80%, within about 85%, within about 90%, within about 95%, or within about 99% of an identical matching. A match may be determined when the corresponding characteristics are within the tolerance and a non-match may be determined when the corresponding characteristics are within the tolerance. Determining a match or non-match between signals can additionally or alternatively include other techniques, such as cross-correlation, or pattern recognition, in any domains, such as the time domain or frequency domain. In some implementations, for example in certain instances of FIGS. 3A and 3B, one or more values derived from the first signal and the second signal (for instance, one signal which is a derivative of the first signal may be compared to another signal which is a derivative of the second signal) can be compared to determine whether the first signal and the second signal match rather than directly comparing the first and second signal.

### Other Variations and Terminology

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed. Additional communication and pairing features are described in Int'l Patent No. PCT/EP2019/064469, the disclosure of which is incorporated herein by reference and may be combined with one of more other features described herein.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method for facilitating communication between two electronic devices, the method comprising:
generating, by a sensor of a first electronic device, a first signal indicative of an ambient environment around the first electronic device;
receiving, by the first electronic device, a second signal from a second electronic device or a third electronic device;
comparing, by the first electronic device, the first signal to the second signal;
determining, by the first electronic device, that the first signal and the second signal match; and
enabling, by the first electronic device, communication between the first electronic device and the second electronic device responsive to the first electronic device determining that the first signal and the second signal match.

2. The method of claim 1, further comprising:
generating the second signal by a sensor of the second electronic device, the second signal being indicative of an ambient environment around the second electronic device; and
transmitting, by the second electronic device, the second signal to the first electronic device,
wherein the receiving the second signal by the first electronic device comprises receiving the second signal from the second electronic device.

3. The method of claim 2, further comprising:
receiving, by the second electronic device, the first signal from the first electronic device;
comparing, by the second electronic device, the second signal to the first signal;
determining, by the second electronic device, that the second signal and the first signal match; and
enabling, by the second electronic device, communication between the second electronic device and the first electronic device responsive to the second electronic device determining that the second signal and the first signal match.

4. The method of claim 1, further comprising:
generating a third signal by a sensor of the second electronic device, the third signal indicative of an ambient environment around the second electronic device;
receiving, by the second electronic device, the second signal from the third electronic device;
comparing, by the second electronic device, the third signal to the second signal;
determining, by the second electronic device, that the third signal and the second signal match; and
enabling, by the second electronic device, communication between the second electronic device and the first electronic device responsive to determining that the third signal and the second signal match,
wherein the receiving the second signal by the first electronic device comprises receiving the second signal from the third electronic device.

5. The method of claim 4, further comprising:
generating the second signal by the third electronic device; and
transmitting, by the third electronic device, the second signal to the first electronic device and the second electronic device.

6. The method of claim 5, further comprising generating and transmitting the first signal by the third electronic device for detection by the first electronic device and the second electronic device, the first signal including a set value within a set frequency band,
wherein the generating the second signal by the third electronic device comprises generating an electromagnetic signal including the set value within the set frequency band.

7. The method of claim 5, further comprising:
receiving, by the third electronic device, the first signal from the first electronic device and the third signal from the second electronic device; and
comparing, by the third electronic device, the first signal and the third signal.

8. The method of claim 4, wherein the receiving the second signal by the first electronic device occurs simultaneously with the receiving the second signal by the second electronic device.

9. The method of claim 4, wherein the generating the first signal by the first electronic device occurs simultaneously with the generating the third signal by the second electronic device.

10. The method of claim 1, wherein the first signal is indicative of electromagnetic radiation in the ambient environment.

11. The method of claim 1, wherein the first signal is indicative of sound in the ambient environment.

12. The method of claim 1, wherein the comparing the first signal to the second signal by the first electronic device comprises comparing a portion of the first signal to a portion of the second signal.

13. The method of claim 1, wherein the comparing the first signal to the second signal by the first electronic device comprises comparing a frequency band in the first signal to a frequency band in the second signal.

14. The method of claim 13, further comprising:
receiving, by the first electronic device, an identification of the frequency band from the third electronic device,
wherein the receiving the second signal by the first electronic device comprises receiving the second signal from the third electronic device.

15. The method of claim 13, further comprising:
transmitting, by the first electronic device, an identification of the frequency band to the third electronic device,
wherein the receiving the second signal by the first electronic device comprises receiving the second signal from the third electronic device.

16. The method of claim 1, further comprising prior to comparing the first signal to the second signal, modifying the first signal to account for a set modification of the ambient environment around the first electronic device by the second electronic device or the third electronic device.

17. The method of claim 1, further comprising transmitting, by the second electronic device or the third electronic device, a modification signal in the ambient environment to modify the first signal generated by the sensor of the first electronic device.

18. (canceled)

19. The method of claim 1, wherein the determining, by the first electronic device, that the first signal and the second signal match comprises determining that the first signal and the second signal have corresponding characteristics within a tolerance.

20. (canceled)

21. A first electronic device comprising:
a first housing;
a first sensor supported by the first housing and configured to generate a first signal indicative of an ambient environment around the first housing;
a first communication interface configured to wirelessly communicate with a second device; and
a first controller supported by the first housing and configured to:
receive a second signal from the second electronic device,
compare the first signal to the second signal,
determine that the first signal and the second signal match, and
enable communication with the second electronic device responsive to the determining that the first signal and the second signal match.

22. A system comprising the first electronic device of claim 21 and the second electronic device, wherein the second electronic device comprises:
a second housing;
a second sensor supported by the second housing and configured to monitor an ambient environment around the second housing;
a second communication interface configured to wirelessly communicate with the first electronic device; and
a second controller supported by the second housing and configured to:
generate the second signal indicative of an ambient environment around the second housing,
receive the first signal from the first electronic device,
compare the second signal to the first signal,
determine that the second signal and the first signal match, and
enable communication with the first electronic device responsive to the determining that the second signal and the first signal match.
